(19) United States
(12) Patent Application Publication
(10) Pub. No.: US 2025/0352719 A1
(43) Pub. Date: Nov. 20, 2025

(54) Portable Mechanical Syringe Pump with Universal Syringe Interface

(71) Applicant: Bio Health Frontiers, Inc., El Dorado Hills, CA (US)

(72) Inventors: Carlos Gutierrez, El Dorado Hills, CA (US); Paul Lambert, El Dorado Hills, CA (US); Travis Schraeder, El Dorado Hills, CA (US); Neal De Beer, El Dorado Hills, CA (US)

(21) Appl. No.: 18/871,800

(22) PCT Filed: Jun. 23, 2022
(86) PCT No.: PCT/US2022/034678

§ 371 Date: Dec. 4, 2024

(51) Int. Cl. A61M 5/142; A61M 5/172; A61M 5/315

(52) U.S. Cl. CPC A61M 5/1424; A61M 5/172; A61M 5/31565

(57) ABSTRACT

A mechanical infusion system includes a housing having top and bottom portions. A pair of sliding slots extend transversely along the bottom portion. A hinge causes the top portion to connect with the bottom portion to form a closed position and separate from the bottom portion to form an open position. The housing forms engagement and syringe receiving cavities to respectively receive a flange and barrel of a syringe. A movable pusher is biased towards the engagement cavity so as to apply force to a plunger of the syringe. The infusion system also includes linkages configured to move transversely in a first direction upon opening of the top portion to cause the pusher to move away from the engagement cavity and move transversely in a second, opposite direction upon closing of the top portion to allow the pusher to retract towards the engagement cavity within the sliding slots.

### TECHNICAL FIELD

The subject matter described herein relates to a mechanically driven syringe pump compatible with different size syringes that is portable and can be handheld.

### BACKGROUND

Healthcare providers use various types and models of syringes made by different manufacturers to infuse drugs or biologics into a patient's body for therapeutic purposes. The variety of syringes used in the healthcare setting are of different size, shape, and features depending on the manufacturer's specifications.

Healthcare providers and users/patients need simple to use and cost-effective mechanical infusion pumps for infusion of drugs or biologics that can be used in the hospital, clinic, or home settings. The infusion pump needs to be portable so the user/patient may ambulate while infusing if needed.

In addition, therapy protocols for primary immunodeficiency administered by subcutaneous injection requires a significant force to be applied to syringe plunger over the duration of the infusion. Ideally, a constant flow rate is desired for the therapy, so a constant force mechanism is required. The force depends on factors including the needle administration set, the drug viscosity, and the prescribed infusion flow rate.

### SUMMARY

An infusion system is provided for use with a syringe having a barrel, flange, and plunger. The infusion system can include various components including a housing having a top portion, a bottom portion, and an aperture. A pair of sliding slots extend transversely along the bottom portion. A hinge causes, in response to user-applied force, the top portion to connect with the bottom portion to form a closed position and separate from the bottom portion to form an open position. The housing forms an engagement cavity having a shape and size to receive the flange of the syringe and a syringe receiving cavity to receive the barrel of the syringe. A movable pusher is positioned within the sliding slots which is biased towards the engagement cavity so as to apply force to the plunger of the syringe when placed in the syringe receiving cavity. The infusion system also includes left and right linkages each having an abutment positioned in one of the respective sliding slots that are configured to (i) move transversely in a first direction upon opening of the top portion to cause the pusher to move away from the engagement cavity and (ii) move transversely in a second, opposite direction upon closing of the top portion to allow the pusher to retract towards the engagement cavity within the sliding slots independent of the left and right linkages.

The pusher can include a spring assembly. Such a spring assembly can take various forms including a rolled-ribbon spring having a spring force ranging, for example, from 1 to 50 pounds force.

The hinge can resist force from the spring assembly during loading of the syringe in the syringe receiving cavity. The hinge can prevent the top portion from inadvertently closing due to force from the spring assembly. The hinge can take various forms including a torque hinge (or two or more torque hinges)

The pusher can include two abutments to each be received within a respective one of the sliding slots such that movement of the left and right linkages causes the pusher to correspondingly move along the sliding slots.

The aperture can have a shape and size to allow either a tip of the syringe to pass therethrough or a tubing set to be coupled to an outlet of the syringe to pass therethrough.

The infusion system can work with syringes of different volumes such as different volumes in a range of 5 to 100 mL.

The top portion can include a viewing window through which progress of an infusion is monitored.

In some variations, the infusion system can include or be used with a variable flow rate controller forming part of an infusion path of the infusion system. The variable flow rate controller can be coupled to a Luer of the syringe. In other variations, the variable flow rate controller can be integrated into the housing.

The subject matter described herein provides many technical advantages. For example, the current subject matter provides healthcare providers and drug manufacturers with a mechanical ambulatory infusion pump that can work with various syringe types, models, and capacities. Further, the current subject matter is advantageous in that it provides a user friendly open-close mechanism that engages/disengages a mechanical spring from the syringe to start or stop the infusion. The size and weight of the mechanical infusion pump allows the user to carry the pump with them for transportation or while infusing by the use of a carrying case that has a shoulder strap.

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

FIG. 1 is a side view of a mechanical infusion pump in a closed position and without a syringe therein;

FIG. 2 is a side view of the mechanical infusion pump of FIG. 1 in a partially open position and without a syringe therein;

FIG. 3 is a side view of the mechanical infusion pump of FIG. 1 in a fully open position and without a syringe therein;

FIG. 4 is an isometric view of the mechanical infusion pump of FIG. 1 in a fully open position and with a syringe placed therein;

FIG. 5 is a side view of the mechanical infusion pump of FIG. 1 in a partially open position and with a syringe placed therein;

FIG. 6 is an isometric view of the mechanical infusion pump of FIG. 1 in a partially open position and with a syringe placed therein;

FIG. 7 is a side view of the mechanical infusion pump of FIG. 1 in a closed position and with a syringe placed therein with a pusher in a first position corresponding to a state in which medication is being delivered;

FIG. 8 is a side view of the mechanical infusion pump of FIG. 1 in a closed position and with a syringe placed therein with a pusher in a second position corresponding to a state in which medication has already been delivered;

FIG. 9 is a first exploded view of certain components of the mechanical infusion pump of FIG. 1;

FIG. 10 is a second exploded view of certain components of the mechanical infusion pump of FIG. 1; and

FIG. 11 is a third exploded view of certain components of the mechanical infusion pump of FIG. 1;

FIG. 12 is a fourth exploded view of certain components of the mechanical infusion pump of FIG. 1; and

FIG. 13 is a fifth exploded view of certain components of the mechanical infusion pump of FIG. 1.

Like reference symbols in the various drawings indicate like elements.

### DETAILED DESCRIPTION

The current subject matter is directed to a mechanical infusion pump that can be used to administer medication housed in syringes having differing sizes/shapes. As will be described in more detail below, the infusion pump can include a universal syringe interface that holds the syringe flanges. For example, in one implementation, the infusion pump can work with multiple syringe models of sizes ranging from 20 to 35 milliliters. In other variations, the syringe volumes can vary from 5 to 100 milliliters. Other size ranges can be adopted (both above 100 milliliters and below 5 milliliters) depending on the desired application.

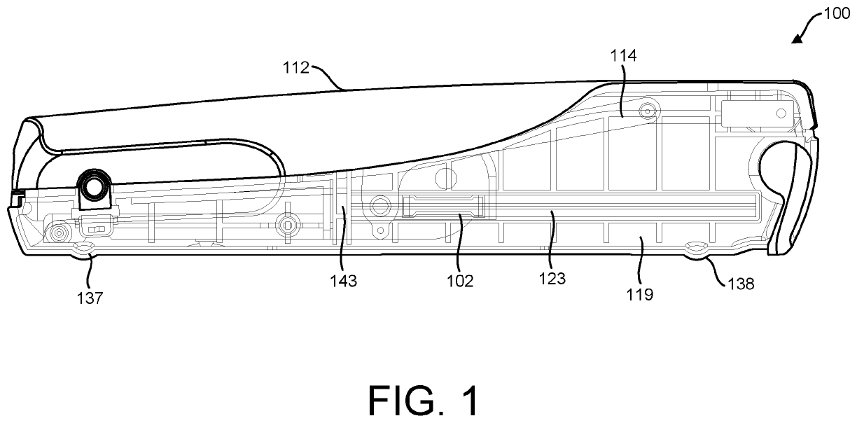

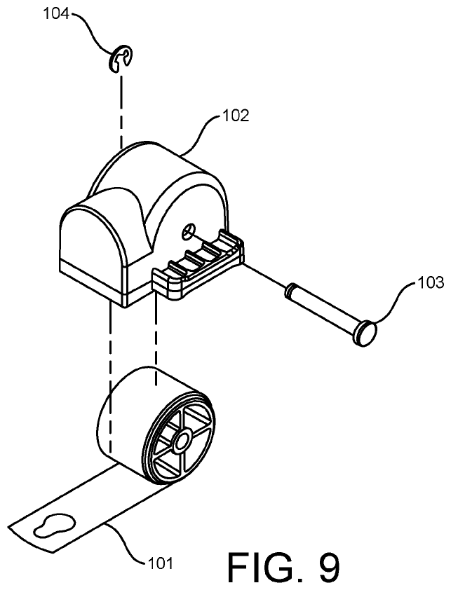

FIGS. 1-8 are diagrams illustrating various aspects of the current mechanical infusion pump details of which are illustrated and further described in connection with FIGS. 9-13 which are exploded views of various components of the mechanical infusion pump. As will be appreciated, certain aspects of the figures are transparent to show the details of the components of the mechanical infusion pump 100 and how they interact with a syringe 130.

FIG. 1 is a diagram that illustrates a side view of the mechanical infusion pump 100 empty in which a top housing 112 is in a closed position. As will be described further, the top housing 112 can couple with a bottom housing which, in some variations, includes a left housing 119 and a right housing 120 which, in turn, can be secured together. The left housing 119 and right housing 120, when connected, can form an engagement cavity 144 that includes an engagement member 143 which has a size and shape to secure a flange 136 of a syringe 130. The left and right housings 119, 120 can have a shape and size (e.g., flat features, etc.) that act to stabilize the infusion pump 100 when it is placed on a horizontal surface. For example, the left and right housings 119, 120 can each include a front foot feature 137 and a back foot feature 138 which include flat surfaces which, in turn, can be used to stabilize the infusion pump 100.

The engagement member 143 can have a size and shape so as to secure flanges 136 of different sizes as might be used in syringes having various volumes ranges such as 20 to 35 milliliters, 5 to 100 milliliters as well as other volumes.

The top housing 112 can be secured to the left housing 119 via a left linkage 114 and a hinge 110 and secured to the right housing 120 via a right linkage 115 and hinge 110. A pusher 102 can be moved along interior sliding slots 123, 124 in response to the linkages 114, 115 being moved away from the engagement cavity 144. The pusher 102 can include a spring mechanism which is biased towards the engagement cavity 144. The spring mechanism can take various forms including a rolled-ribbon spring which, as will be illustrated and described later, acts as a force generating mechanism which provides an approximate constant force over the working range of the spring.

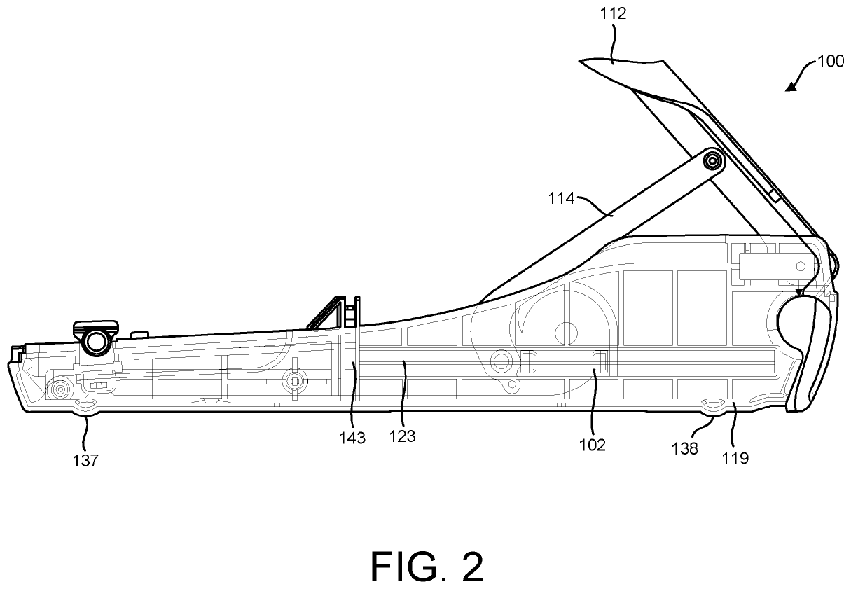

FIG. 2 is a diagram that illustrates a side view of the mechanical infusion pump 100 empty in a halfway open position (i.e., the top housing 112 is not yet fully extended, and consequently, pusher 102 not yet fully retracted). Note that the top housing 112 is truncated in FIGS. 2-6 for illustrative purposes. The size of the mechanical infusion pump is such that it can be handheld so that the top housing 112 can be selectively opened or closed by a user holding the bottom housing in one hand and opening the top housing 112 in the user's other hand. The act of opening the top housing 112 causes the connected linkages 114, 115 to move away from the engagement cavity 144 which, in turn, causes the pusher 102 to also move away from the engagement cavity 144.

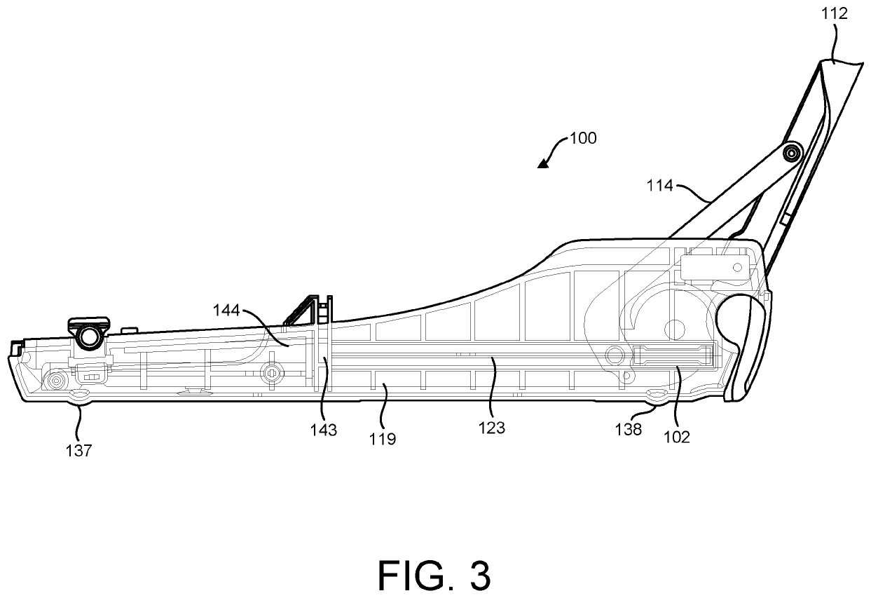

FIG. 3 is a diagram that illustrates a side view of the mechanical infusion pump 100 empty and in a fully open position (i.e., the top housing is fully extended 112) in which the pusher 102 is fully extended so that a pre-filled syringe 130 can be placed within the mechanical infusion pump 100.

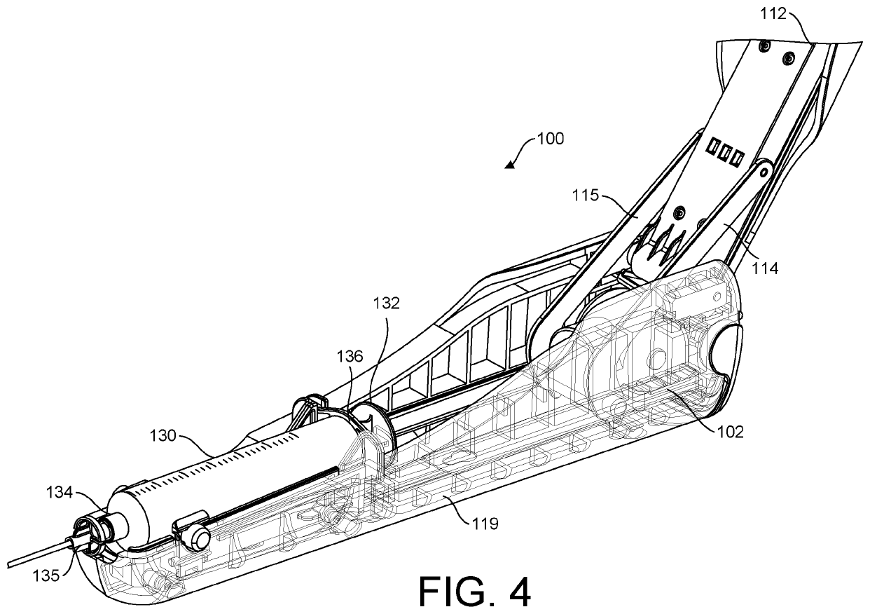

FIG. 4 is a diagram that illustrates a side view of the mechanical infusion pump 100 with a syringe 130 placed therein while the mechanical infusion pump 100 is a fully open position. The pusher 102, in this position, is not in contact with the plunger 132 of the syringe 130 to allow the syringe 130 to be loaded into the mechanical infusion pump. As will be described in further detail below, the pusher 102 is configured to apply force to the plunger 132 which, in turn, causes, fluid within the syringe 130 to be expelled from the outlet 134 of the syringe.

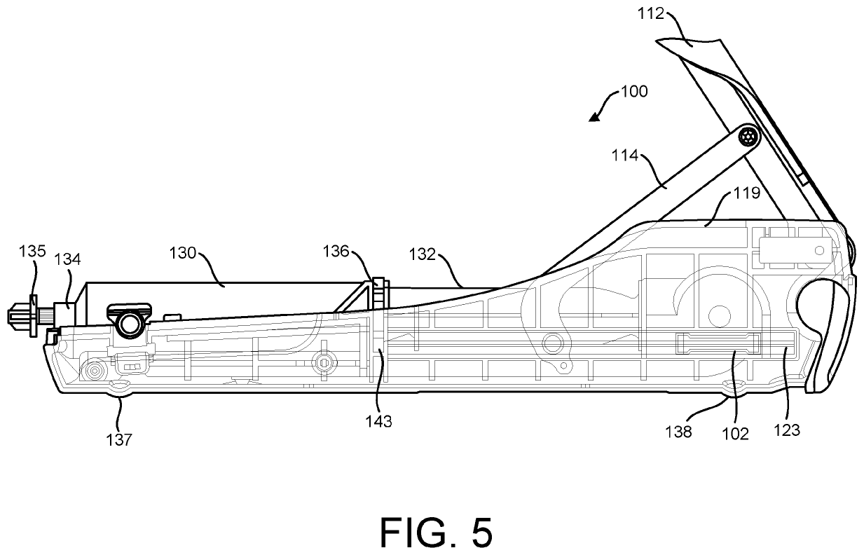

FIG. 5 is a diagram that illustrates the mechanical infusion pump 100 with a syringe 130 placed therein while it is being transitioned from a fully open position to a closed position. Here, the pusher 102 (which is biased towards the engagement cavity 144) is in contact with the plunger 132 of the syringe due to the top housing 112 being moved towards the closed position (which in turn causes the respective linkages 114, 115 to advance towards the engagement cavity 144).

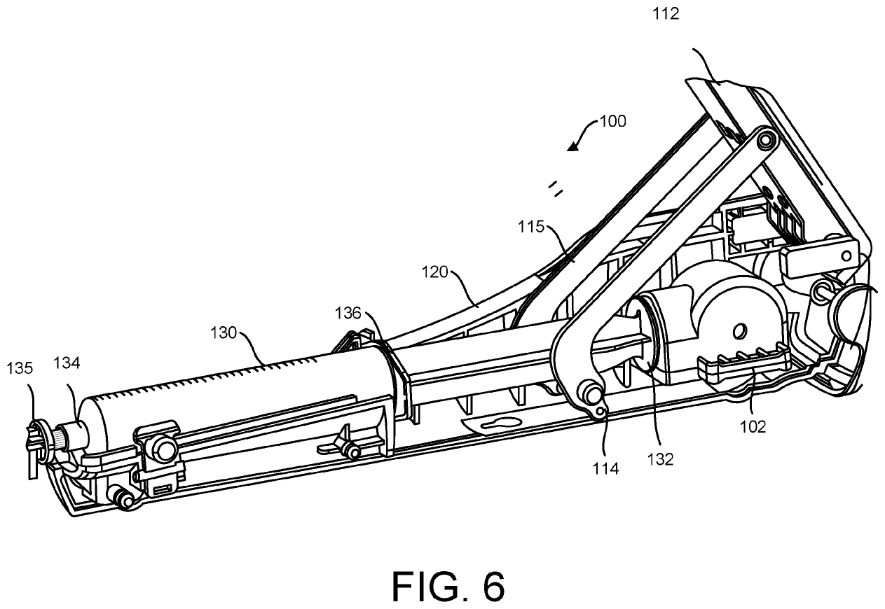

FIG. 6 is an isometric view of the mechanical infusion pump 100 illustrating the pusher 102 applying force to the plunger 132 of the syringe 130.

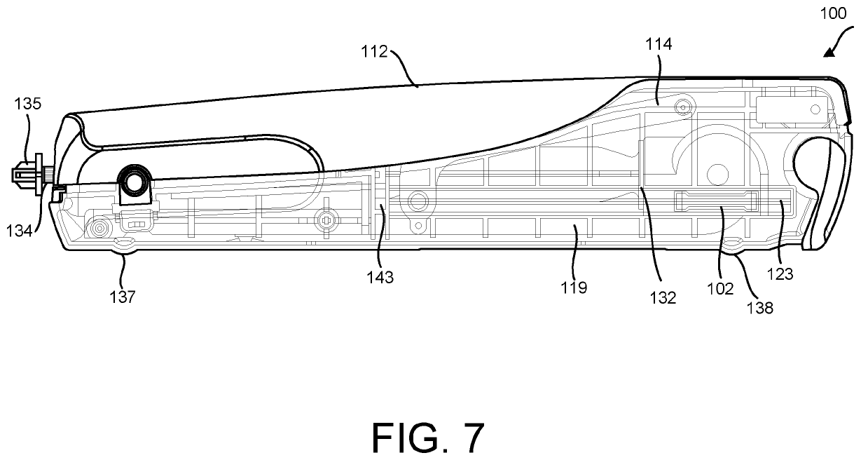

FIG. 7 is a diagram illustrating the mechanical infusion pump 100 in a closed position in which the pusher 102 is applying force to the plunger 132 which, in turn, causes medication to be expelled from an outlet 134 of the syringe 130 (which can be, for example, coupled to or otherwise connecting to a tubing set 135 to deliver medication to a patient). The outlet 134 of the syringe 130 can, in some variations, include a Luer lock to secure the tubing set 135 and the like. Various type of tubing sets can be used including those that provide a constant or metered flow rate over a range of force applied to the plunger 132 of the syringe. In some variations, a variable flow rate controller can form part of an infusion path of the infusion system. Such a flow rate controller can be integrated into the mechanical infusion pump 100, in some variations, while in other variations, it is connectable to the Luer lock of the syringe 130 and/or the tubing set 135. Example controllers including the VERSARATE and VERSARATE PLUS adjustable flow rate controllers by EMED Technologies of El Dorado Hills, California.

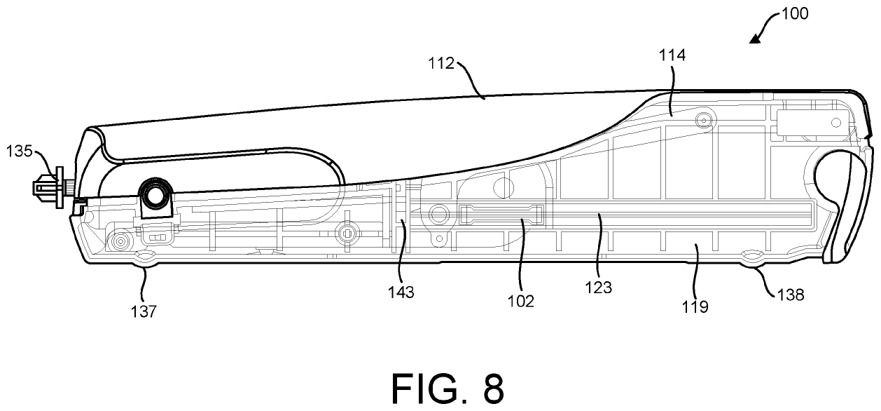

FIG. 8 is a diagram illustrating the top housing 112 of the mechanical infusion pump 100 being completely closed and the medication in the syringe as being fully expelled out the outlet 134. In this situation, the pusher 102 has advanced towards the outlet 134 of the syringe 130 via a pair of sliding slots 123, 124 (not shown) respectively integrated into the left housing 119 and the right housing 120.

FIG. 9 illustrates a spring assembly 101, a pusher 102, and a pin 103 (e.g., a grooved Clevis pin, etc.) and a clip 104 (e.g., an E-clip) are provided. The spring assembly 101 within the pusher 102 is such that it is biased so as to be coiled. A screw 106 can pass through a notch in the spring assembly 101 to secure the spring assembly 101 to the spring block 105 which in turn is secured to the left and right housings 119, 120.

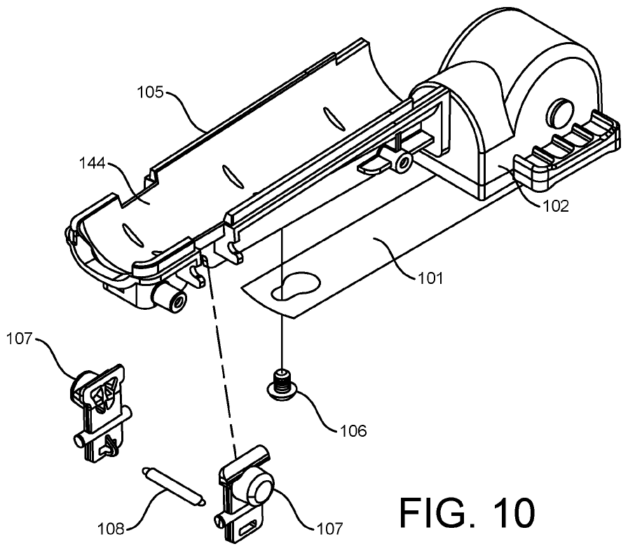

With reference to FIG. 10, a spring block 105, a screw 106, buttons 107, and an extension spring 108 are provided. The spring block 105 can have a size and shape so as to receive a barrel portion of the syringe 130 and, when the mechanical infusion pump 100 is assembled, can form a syringe receiving cavity extending from an aperture through which an outlet 134 of the syringe 130 extends and configured to receive a barrel of the syringe 130. The screw 106 can secure the spring assembly 101 to a bottom side of the spring block 105. The buttons 107 can be assembled to the spring block 105 and left and right housings 119, 120. The extension spring 108 can be connected between the two buttons to bias them in one direction. The top housing 112 can interface with the buttons 107 when the top housing 112 is in the closed position by way of latches (or other abutments). The buttons 107 can be configured so as to secure the top housing 112 in the closed position. The top housing 112 can be opened when the user presses both buttons 107 inwards, thereby releasing the latches and allowing the top housing 112 to transition to the open position.

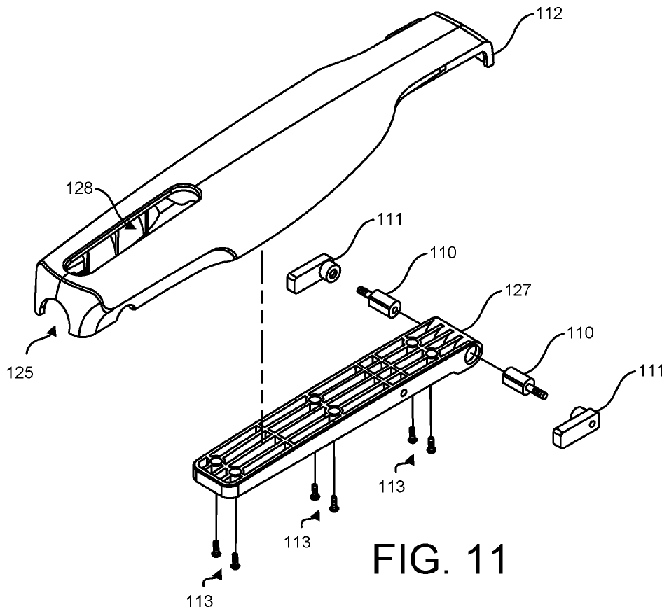

With reference to FIG. 11, the top housing 112 can be secured to a frame 127 by way of torque hinges 110 which can be secured to the housing via the hinge adapters 111. Similarly, screws 113 can be used to secure the frame 127 to other components forming part of the mechanical infusion pump 100. The torque hinge 110 can be a friction torque hinge that resists the force of the spring assembly 101 during the syringe 130 loading state. This aspect improves the user experience, so they are not struggling to fight the force of the spring assembly 101. The torque hinge 110 can also prevent the top housing 112 from inadvertently closing due to the force of the spring assembly 101 (i.e., the torque hinge 110 counteracts the bias of the spring assembly 101). In addition, the top housing 112 can include a viewing window 128 to allow a user to see the advancement of the plunger of the syringe (i.e., the user can monitor the infusion progress, etc.). The top housing 112 can also include an aperture 125 through which the outlet 134 of the syringe 130 can protrude and/or be accessed.

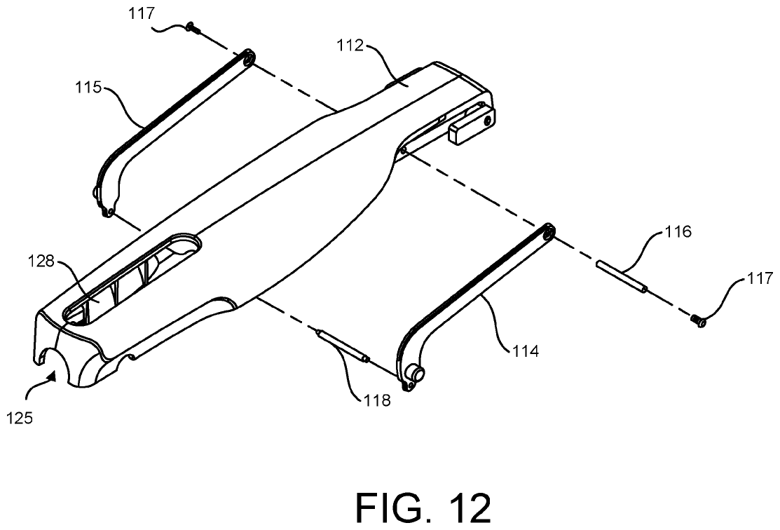

With reference to FIG. 12, the left linkage 114 and right linkage 115 can be secured to the top housing 112 by way of a link connector 116, a screw 117 (e.g., a flanged button screw, etc.), and a link pin 118. Other securing mechanisms can be alternatively used such as a press fit pin.

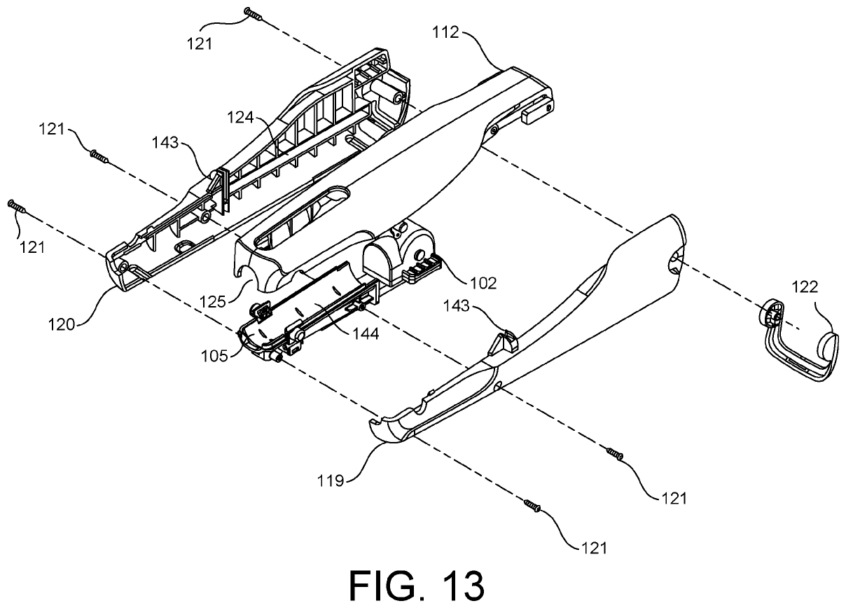

With reference to FIG. 13, the left housing 119 and the right housing 120 can be secured to each other by means of a series of screws 121 (e.g., a self-tapping plastic screw, etc.) in some variations. In other variations, other securing mechanisms such as snap-in tab features molded in the parts can be utilized to secure the left housing 119 to the right housing 120. Optionally, the mechanical infusion pump 100 can include a hanger 122 which can allow a hanging point on the mechanical infusion pump 100. The hanger 122 can be used to place the mechanical infusion pump on an IV pole or other protrusion.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

1. An infusion system for use with a syringe having a barrel, flange, and plunger, the system comprising:
a housing having a top portion, a bottom portion, and an aperture;
a pair of sliding slots extending transversely along the bottom portion;
a hinge configured to cause, in response to user-applied force, the top portion to connect with the bottom portion to form a closed position and separate from the bottom portion to form an open position;
an engagement cavity formed by the housing having a shape and size to receive the flange of the syringe; a syringe receiving cavity formed by the housing extending configured to receive the barrel of the syringe;
a movable pusher positioned within the sliding slots, the movable pusher being biased towards the engagement cavity so as to apply force to the plunger of the syringe when placed in the syringe receiving cavity; and
left and right linkages each having an abutment positioned in a respective one of the sliding slots that are configured to (i) move transversely in a first direction upon opening of the top portion to cause the pusher to move away from the engagement cavity and (ii) move transversely in a second, opposite direction upon closing of the top portion to allow the pusher to retract towards the engagement cavity within the sliding slots independent of the left and right linkages.

2. The infusion system of claim 1, wherein the pusher comprises a spring assembly.

3. The infusion system of claim 2, wherein the spring assembly comprises a rolled-ribbon spring having a spring force ranging from 1 to 50 pounds force.

4. The infusion system of claim 2 wherein the hinge resists force from the spring assembly during loading of the syringe in the syringe receiving cavity.

5. The infusion system of claim 2, wherein the hinge prevents the top portion from inadvertently closing due to force from the spring assembly.

6. The infusion system of claim 5, wherein the hinge is a torque hinge.

7. The infusion system of claim 1, wherein the pusher comprises two abutments to each be received within a respective one of the sliding slots such that movement of the left and right linkages causes the pusher to correspondingly move along the sliding slots.

8. The infusion system of claim 1 wherein the aperture has a shape and size to allow either a tip of the syringe to pass therethrough or a tubing set to be coupled to an outlet of the syringe to pass therethrough.

9. The infusion system of claim 1, wherein the syringe has a volume in range of 5 to 100 mL.

10. The infusion system of claim 1 wherein the top portion comprising a viewing window through which progress of an infusion is monitored.

11. The infusion system of claim 1 further comprising: a variable flow rate controller forming part of an infusion path of the infusion system.

12. The infusion system of claim 11, wherein the variable flow rate controller is coupled to a Luer of the syringe.

13. The infusion system of claim 11, wherein the variable flow rate controller is integrated into the housing.

14. An infusion system comprising:
hinge means for causing, in response to user-applied force, a top portion to connect with the bottom portion to form a closed position and separate from a bottom portion to form an open position; engagement means having a shape and size to receive and secure a syringe;
pushing means biased towards the engagement means so as to apply force to the syringe; and
linkage means to (i) move transversely in a first direction upon opening of the top portion to cause the pushing means to move away from the engagement means and (ii) move transversely in a second, opposite direction upon closing of the top portion to allow the pushing means to retract towards the engagement means.

15. A system comprising:
a syringe having a barrel, flange, and plunger;
a housing having a top portion, a bottom portion, and an aperture;
a pair of sliding slots extending transversely along the bottom portion;
a hinge configured to cause, in response to user-applied force, the top portion to connect with the bottom portion to form a closed position and separate from the bottom portion to form an open position;
an engagement cavity formed by the housing having a shape and size to receive the flange of the syringe;
a syringe receiving cavity formed by the housing extending configured to receive the barrel of the syringe;
a movable pusher positioned within the sliding slots, the movable pusher being biased towards the engagement cavity so as to apply force to the plunger of the syringe when placed in the syringe receiving cavity; and
left and right linkages each having an abutment positioned in a respective one of the sliding slots that are configured to (i) move transversely in a first direction upon opening of the top portion to cause the pusher to move away from the engagement cavity and (ii) move transversely in a second, opposite direction upon closing of the top portion to allow the pusher to retract towards the engagement cavity within the sliding slots independent of the left and right linkages.
